(19) United States
(12) Patent Application Publication
(10) Pub. No.: US 2011/0009447 A1
(43) Pub. Date: Jan. 13, 2011

(54) Substituted Benzoic Acid Amides and Use thereof for the Inhibition of Angiogenesis

(76) Inventors: Andreas Huth, Berlin (DE); Dieter Seidelmann, Berlin (DE); Karl-Heinz Thierauch, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/258,345

(22) PCT Filed: Apr. 24, 2001
(86) PCT No.: PCT/EP01/04627

§ 371 Date: Nov. 3, 2009

(51) Int. Cl. A61K 31/4725; C07D 213/56; C07D 401/12; C07D 403/12; C07D 409/12; C07C 235/56; A61K 31/4409; A61K 31/444; A61K 31/4709; A61K 31/4439; A61K 31/4436; A61K 31/167; A61P 19/02; A61P 35/00; A61P 17/06; A61P 27/06; A61P 27/02; A61P 13/12; A61P 37/06; A61P 9/10

(52) U.S. Cl. 514/310; 546/337; 546/264; 546/143; 546/162; 546/277.4; 546/280.4; 564/174; 514/357; 514/332; 514/313; 514/339; 514/337; 514/622

(57) ABSTRACT

Substituted benzoic acid amides of formula (I) and their use as pharmaceutical agents for treating diseases that are triggered by persistent angiogenesis as well as their intermediate products for the production of benzoic acid amides are described.

The invention relates to substituted benzoic acid amides and their use as pharmaceutical agents for treating diseases that are triggered by persistent angiogenesis as well as their intermediate products for the production of benzoic acid amides.

Persistent angiogenesis can be the cause of various diseases, such as psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases and arteriosclerosis or can result in an aggravation of these diseases.

Direct or indirect inhibition of the VEGF receptor (VEGF=vascular endothelial growth factor) can be used for treating such diseases and other VEGF-induced pathological angiogenesis and vascular permeable conditions, such as tumor vascularization. For example, it is known that the growth of tumors can be inhibited by soluble receptors and antibodies against VEGF.

Persistent angiogenesis is induced by the factor VEGF via its receptor. So that VEGF can exert this action, it is necessary that VEGF bind to the receptor, and a tyrosine phosphorylation is induced.

Only derivatives of the compounds claimed here that have been removed were described as calpain inhibitors (WO 9823581, WO 9825883), phospholipase A2 inhibitors (WO 9700583), prostaglandin D2 antagonists (WO 9700853), neurokinin A antagonists (WO 95 16682), tranquilizers (U.S. Pat. No. 3,892,752) or anorexigenics (FR 1600541).

An action of these known compounds in connection with VEGF was not previously described.

It has now been found that compounds of general formula I

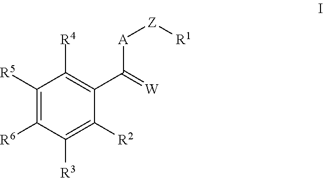

in which
A stands for the group ═NR⁷,
W stands for oxygen, sulfur, two hydrogen atoms or the group ═NR⁸,
Z stands for a bond, the group ═NR¹⁰ or ═N—, for branched or unbranched C₁₋₁₂-alkyl or for the group

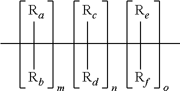

m, n and o stand for 0-3,
Rₐ, R_{b}, R_{c}, R_{d}, Rₑ, R_{f}, independently of one another, stand for hydrogen, fluorine, C₁₋₄-alkyl or the group ═NR¹⁰, and/or Rₐ and/or R_{b} can form a bond with R^{c}, and/or R_{d} or R_{c} can form a bond with Rₑ and/or R_{f}, or up to two of radicals Rₐ-R_{f} can close a bridge with up to 3 C atoms each to form R¹ or to form R⁷,
R¹ stands for branched or unbranched C₁₋₆-alkyl, C₂₋₁₂-alkenyl or C₃₋₁₂-alkinyl that is optionally substituted in one or more places with halogen or C₁₋₆-alkyl or for C₃₋₁₀-cycloalkyl or C₃₋₁₀-cycloalkenyl that is optionally substituted in one or more places with halogen or C₁₋₆-alkyl, or for aryl or heteroaryl that is unsubstituted or that is optionally substituted in one or more places with halogen, C₁₋₆-alkyl, C₁₋₆-alkoxy, or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkinyl,
R¹¹ means monocyclic aryl, bicyclic aryl or heteroaryl that is unsubstituted or that is optionally substituted in one or more places with halogen, C₁₋₆-alkyl, C₁₋₆-alkoxy, or hydroxy,
R⁴, R⁵ and R⁶ stand for hydrogen, halogen, or C₁₋₆-alkoxy, C₁₋₆-alkyl or C₁₋₆-carboxyalkyl that is unsubstituted or that is optionally substituted in one or more places with halogen,
   or R⁴ and R⁵ together form the group

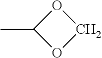

R⁷ stands for hydrogen or C₁₋₆-alkyl or forms a bridge with up to 3 ring members with Rₐ-R_{f} from Z or to form R¹,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, and R¹ must not be thiazole, as well as isomers and salts thereof,
   stop tyrosine phosphorylation or the persistent angiogensis and thus prevent the growth and propagation of tumors.

If R⁷ forms a bridge to R¹, heterocyclic compounds result to which R¹ is fused. For example, there can be mentioned:

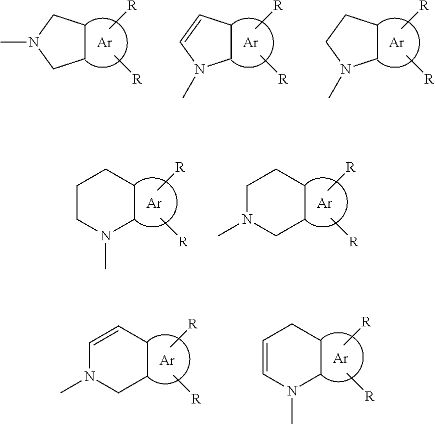

If Rₐ, R_{b}, R_{c}, R_{d}, Rₑ, R_{f}, independently of one another, represent hydrogen or C₁₋₄ alkyl, Z thus forms an alkyl chain.

If Rₐ, and/or R_{b} form a bond with R_{c} and/or R_{d} or R_{c} and/or R_{d} form a bond with Rₑ and/or R_{f}, Z stands for an alkenyl or alkinyl chain.

If Rₐ-R_{f} forms a bridge with itself, Z represents a cycloalkyl or cycloalkenyl group.

If up to two of radicals Rₐ-R_{f} form a bridge with up to 3 C atoms to form R¹, Z together with R¹ is a benzocondensed or hetaryl-condensed (Ar) cycloalkyl.

For example, there can be mentioned:

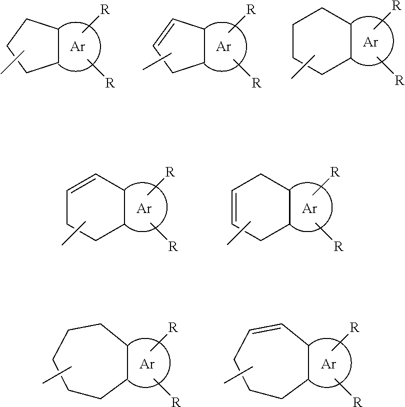

If one of radicals Rₐ-R_{f} closes a bridge to form R⁷, a nitrogen heterocyclic compound is formed that can be separated from R¹ by a group.

For example, there can be mentioned:

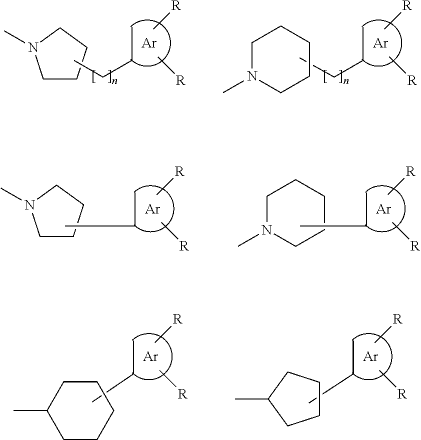

Alkyl is defined in each case as a straight-chain or branched alkyl radical, such as, for example, methyl, ethyl, propyl, isopropyl, butyl, isobutyl, sec-butyl, pentyl, isopentyl, or hexyl, whereby C₁₋₄-alkyl radicals are preferred.

Cycloalkyl is defined in each case as cyclopropyl, cyclobutyl, cyclopentyl, cyclohexyl, or cycloheptyl, cyclooctyl, cyclononyl or cyclodecyl.

Cycloalkenyl is defined in each case as cyclobutenyl, cylopentenyl, cyclohexenyl, cycloheptenyl, cyclooctenyl, cyclononenyl or cyclodecenyl, whereby the linkage can be carried out both to the double bond and to the single bonds.

Halogen is defined in each case as fluorine, chlorine, bromine or iodine.

The alkenyl and alkinyl substituents are in each case straight-chain or branched and contain 2-6, preferably 2-4 C atoms. For example, the following radicals can be mentioned: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1 but-1-en-3-yl, ethinyl, prop-1-in-1-yl, but-1-in-1-yl, but-2-in-1-yl, but-3-en-1-yl, and allyl.

The aryl radical in each case has 6-12 carbon atoms, such as, for example, naphthyl, biphenyl and especially phenyl.

The heteroaryl radical in each case can be benzocondensed. For example, as 5-ring heteroaromatic compounds, there can be mentioned; thiophene, furan, oxazole, thiazole, imidazole, and benzo derivatives thereof, and as 6-ring heteroaromatic compounds, there can be mentioned: pyridine, pyrimidine, triazine, quinoline, isoquinoline and benzo derivatives.

The aryl radical and the heteroaryl radical in each case can be substituted in the same way or differently in 1, 2 or 3 places with halogen, C₁₋₄-alkoxy, nitro, trifluoromethyl, trifluoromethoxy, cyano, SO_{q}R⁵ or C₁₋₄-alkyl, whereby q stands for 0-2.

If an acid group is included, the physiologically compatible salts of organic and inorganic bases are suitable as salts, such as, for example, the readily soluble alkali salts and alkaline-earth salts as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, fumaric acid, i.a.

Those compounds of general formula I in which
A stands for the group ═NR⁷,
W stands for oxygen, sulfur or two hydrogen atoms,
Z stands for a bond, the group ═NR¹⁰ or for branched or unbranched C₁₋₁₂-alkyl,
R¹ stands for branched or unbranched C₁₋₆-alkyl that is optionally substituted in one or more places with halogen or C₁₋₆-alkyl; or for C₃₋₁₀-cycloalkyl that is optionally substituted in one or more places with halogen or C₁₋₆-alkyl, or for phenyl, pyridyl, naphthyl, quinolyl, isoquinolyl, indanyl, tetralinyl, indolyl, thienyl, indazolyl or benzothiazolyl that is unsubstituted or that is optionally substituted in one or more places with halogen, C₁₋₆-alkyl, C₁₋₆-alkoxy or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkinyl,
R¹¹ means phenyl, pyrimidinyl or pyridyl that is unsubstituted or that is optionally substituted in one or more places with halogen, C₁₋₆-alkoxy or hydroxy,
R⁴, R⁵, R⁶ and R⁷ stand for hydrogen,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, as well as isomers and salts thereof,
   have proven especially effective.

Those compounds of general formula I in which
A stands for the group ═NR⁷,
W stands for oxygen, or for one or two hydrogen atoms,
Z stands for a bond, the group ═NR¹⁰ or for branched or unbranched C₁₋₁₂-alkyl,

R¹ stands for branched or unbranched C₁₋₆-alkyl, or for C₃₋₁₀-cycloalkyl that is optionally substituted in one or more places with halogen or C₁₋₆-alkyl; or for phenyl, pyridyl, naphthyl, quinolyl, isoquinolyl, indenyl, tetralinyl, indolyl, indazolyl, benzothiazolyl or thienyl that is unsubstituted or that is optionally substituted in one or more places with halogen, C₁₋₆-alkyl, C₁₋₆-alkoxy or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkinyl,
R¹¹ stands for phenyl, pyrimidinyl or pyridyl that is unsubstituted or that is optionally substituted in one or more places with halogen, C₁₋₆-alkoxy or hydroxy,
R⁴, R⁵, R⁶ and R⁷ stand for hydrogen,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, as well as isomers and salts thereof, have proven quite especially effective.

The compounds according to the invention prevent a phosphorylation, i.e., certain tyrosine kinases can be selectively inhibited, whereby the persistent angiogenesis can be stopped. Thus, for example, the growth and the propagation of tumors is prevented.

The compounds of general formula I according to the invention also contain the possible tautomeric forms and comprise the E- or Z-isomers or, if a chiral center is present, also the racemates and enantiomers.

The compounds of formula I as well as their physiologically compatible salts can be used as pharmaceutical agents based on their inhibitory activity relative to the phosphorylation of the VEGF receptor. Based on their profile of action, the compounds according to the invention are suitable for treating diseases that are caused by persistent angiogenesis.

Since the compounds of formula I are identified as inhibitors of the tyrosine kinase KDR and FLT, they are suitable in particular for treating those diseases that are caused by persistent angiogenesis that is triggered via the VEGF receptor or by an increase in vascular permeability.

The subject of this invention is also the use of the compounds according to the invention as inhibitors of the tyrosine kinase KDR and FLT.

Subjects of this invention are thus also pharmaceutical agents for treating tumors or use thereof.

The compounds according to the invention can be used either alone or in a formulation as pharmaceutical agents for treating psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis and injuries to nerve tissue.

In treating injuries to nerve tissue, quick scar formation on the injury sites can be prevented with the compounds according to the invention, i.e., scar formation is prevented from occurring before the axons reconnect. A reconstruction of the nerve compounds was thus facilitated.

The formation of ascites in patients can also be suppressed with the compounds according to the invention. VEGF-induced edemas can also be suppressed.

Such pharmaceutical agents, their formulations and uses, are also subjects of this invention.

The invention thus also relates to the use of compounds of general formula I for the production of a pharmaceutical agent for treating tumors, psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, and injuries to nerve tissue.

To use the compounds of formula I as pharmaceutical agents, the latter are brought into the form of a pharmaceutical preparation, which in addition to the active ingredient for enteral or parenteral administration contains suitable pharmaceutical, organic or inorganic inert carrier materials, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, capsules or in liquid form, for example as solutions, suspensions or emulsions. They optionally contain, moreover, adjuvants such as preservatives, stabilizers, wetting agents or emulsifiers, salts for changing osmotic pressure or buffers.

For parenteral administration, especially injection solutions or suspensions, especially aqueous solutions of the active compounds in polyhydroxyethoxylated castor oil, are suitable.

As carrier systems, surface-active adjuvants such as salts of bile acids or animal or plant phospholipids, but also mixtures thereof as well as liposomes or components thereof can also be used.

For oral administration, especially tablets, coated tablets or capsules with talc and/or hydrocarbon vehicles or binders, such as for example, lactose, corn starch or potato starch, are suitable. The administration can also be carried out in liquid form, such as, for example, as juice, to which optionally a sweetener is added.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into 2 or more daily doses.

The above-described formulations and forms for dispensing are also subjects of this invention.

The production of the compounds according to the invention is carried out according to methods that are known in the art. For example, compounds of formula I are obtained, in that

a) in a compound of general formula II

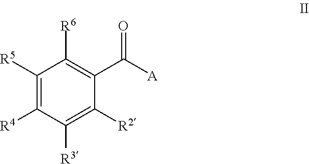

in which R⁴ to R⁶ have the above-mentioned meaning and A is halogen or OR¹¹, whereby R¹¹ means hydrogen, C₁₋₄-alkyl or C₁₋₄-acyl, and R^{2′} and R^{3′} mean hydrogen, aldehyde, halogen or OH, O-triflate, O-tosylate or O-mesylate, first R^{2′} or R^{3′} is converted into an alkenyl or alkinyl, optionally saturated in the corresponding alkane, and then COA is converted into an amide,

or

b) a compound of general formula III

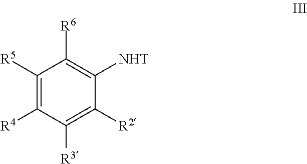

in which R⁴ to R⁶ have the above-mentioned meaning and T means a protective group, is acylated and then optionally the keto group is reduced to alcohol or alkane, the protective group is cleaved off, the amine is converted into a nitrile, and the nitrile is saponified and converted into an amide.

The sequence of steps can be interchanged in all cases.

The amide formation is carried out according to methods that are known in the literature.

For amide formation, it is possible to start from a corresponding ester. The ester is reacted according to J. Org. Chem. 1995, 8414 with aluminum trimethyl and the corresponding amine in solvents such as toluene at temperatures of 0° C. up to the boiling point of the solvent. If the molecule contains two ester groups, both are converted into the same amide.

When nitriles are used instead of ester, amidines are obtained under analogous conditions.

For amide formation, however, all processes that are known from peptide chemistry are also available. For example, the corresponding acid can be reacted with the amine in aprotic polar solvents, such as, for example, dimethylformamide, via an activated acid derivative that can be obtained with, for example, hydroxybenzotriazole and a carbodiimide, such as, for example, diisopropylcarbodiimide, or else with preformed reagents, such as, for example, HATU (Chem. Comm. 1994, 201) or BTU, at temperatures of between 0° C. and the boiling point of the solvent, preferably at 80° C. For the amide formation, the process can also be used with the mixed acid anhydride, imidazolide or azide.

Salicylamides are obtained if the corresponding phenol is reacted in the presence of a Friedel-Crafts catalyst, such as boron trichloride, with isocyanates or isothiocyanates in solvents, such as, for example, toluene, at temperatures of 0° C. up to the boiling point of the solvent.

If various amide groups are to be introduced into the molecule, for example, the second ester group must be introduced into the molecule after the production of the first amide group and then amidated, or there is a molecule in which one group is present as an ester, the other is present as an acid, and the two groups are amidated in succession according to various methods.

Thioamides can be obtained from the anthranilamides by reaction with diphosphadithianes according to Bull Soc. Chirp. Belg. 87, 229, 1978 or by reaction with phosphorus pentasulfide in solvents such as pyridine or even quite without solvent at temperatures of 0° C. to 200° C.

The products can also be subjected to an electrophilic aromatic substitution. The substitution then takes place on compounds of formula III in the ortho- or para-position into the or one of the amino group(s, into compounds of formula II in the meta-position) to form the carbonyl group. Thus, acylation can be done by Friedel-Crafts acylation with acid chlorides in the presence of Friedel-Crafts catalysts, such as, for example, aluminum trichloride in solvents such as nitromethane, carbon disulfide, methylene chloride or nitrobenzene at temperatures of between 0° C. and the boiling point of the solvent, preferably at room temperature. According to processes that are known in the literature, one or more nitro groups can be introduced without solvent, for example by nitrating acid, nitric acid of various concentrations, or by metal nitrates, such as, for example, copper(II) nitrate or iron(III) nitrate in polar solvents such as ethanol or glacial acetic acid or else in acetic anhydride.

The introduction of halogens is carried out according to processes that are known in the literature, e.g., by reaction with bromine, N-bromo- or N-iodosuccinimide or utropin hydrotribromide in polar solvents such as tetrahydrofuran, acetonitrile, methylene chloride, glacial acetic acid or dimethylformamide.

The reduction of the nitro group is performed in polar solvents at room temperature or elevated temperature. As catalysts for the reduction, metals such as Raney nickel or noble-metal catalysts such as palladium or platinum or else palladium hydroxide optionally on vehicles are suitable. Instead of hydrogen, for example, ammonium formate, cyclohexene or hydrazine can also be used in a known way. Reducing agents such as tin(II) chloride or titanium(III) chloride can also be used, such as complex metal hydrides, optionally in the presence of heavy metal salts. Iron can also be used as a reducing agent. The reaction is then performed in the presence of an acid, such as, e.g., acetic acid or ammonium chloride, optionally with the addition of a solvent, such as, for example, water, methanol, iron/ammonia, etc. With an extended reaction time, an acylation of the amino group can occur in this variant.

If an alkylation of an amino group is desired, alkylation can be done according to commonly used methods—for example with alkyl halides—or according to the Mitsonubo variant by reaction with an alcohol in the presence of, for example, triphenylphosphine and azodicarboxylic acid ester. The amine can also be subjected to reductive alkylation with aldehydes or ketones, whereby the reaction is performed in the presence of a reducing agent, such as, for example, sodium cyanoborohydride in a suitable inert solvent, such as, for example, ethanol, at temperatures of 0° C. up to the boiling point of the solvent. If a start is made from a primary amino group, the reaction can be performed optionally in succession with two different carbonyl compounds, whereby mixed derivatives are obtained [Literature, e.g., Verardo et al. Synthesis (1993), 121; Synthesis (1991), 447; Kawaguchi, Synthesis (1985), 701; Micovic et al. Synthesis (1991), 1043]. It can be advantageous first to form the Schiff base by reaction of the aldehyde with the amine in solvents such as ethanol or methanol, optionally with the addition of adjuvants such as glacial acetic acid, and then to add only reducing agents, such as, e.g., sodium cyanoborohydride.

The hydrogenation of alkene or alkine groups in the molecule is carried out in the usual way by, for example, catalytically activated hydrogen. As catalysts, heavy metals such as palladium or platinum, optionally on a vehicle, or Raney nickel can be used. The procedure is performed at temperatures of 0° C. up to the boiling point of the solvent and at pressures of up to 20 bar, but preferably at room temperature and normal pressure. By the use of catalysts, such as, for example, a Lindlar catalyst, triple bonds can be partially hydrogenated to double bonds, whereby preferably the Z-form is produced. This hydrogenation is preferably performed in pyridine as a solvent with palladium on calcium carbonate as a catalyst. In the same way, the Z-double bond can be produced from the triple bond by reduction with diimine, produced, for example, according to R. M. Moriatry et al. Synth. Comm. 17, 703, 1987.

The acylation of an amino group is carried out in the usual way, with, for example, acid halide or acid anhydride optionally in the presence of a base such as dimethylaminopyridine in solvents such as methylene chloride, tetrahydrofuran or pyridine, according to the Schotten-Baumann variant in aqueous solution at weakly alkaline pH or by reaction with an anhydride in glacial acetic acid.

A reduction of a keto group is carried out according to processes that are known in the art. Thus, by complex metal hydrides, such as, for example, sodium borohydride in solvents such as methanol or isopropanol, the keto group, in addition to the amide group or ester group, can be reduced selectively to alcohol. A reduction of a keto group to the methylene group can be carried out according to Clemmensen with zinc in hydrochloric acid or else, for example, with silanes in trifluoroacetic acid.

The introduction of the halogens chlorine, bromine, iodine or the azido group via an amino group can be carried out, for example, also according to Sandmeyer by the diazonium salts that are intermediately formed with nitrites being reacted with copper(I) chloride or copper(I) bromide in the presence of the corresponding acid, such as hydrochloric acid or hydrobromic acid or with potassium iodide.

If an organic nitrite is used, the halogens can be introduced into a solvent, such as, for example, dimethylformamide, e.g., by adding methylene iodide or tetrabromomethane. The removal of the amino group can be achieved either by reaction with an organic nitrite in tetrahydrofuran or by diazotization and reductive boiling-down of the diazonium salt with, for example, phosphorous acid, optionally with the addition of copper(I) oxide.

The introduction of fluorine can be accomplished, for example, by Balz-Schiemann reaction of the diazonium tetrafluoroborate or according to J. Fluor. Chem. 76, 1996, 59-62 by diazotization in the presence of HFxpyridine and subsequent boiling-down optionally in the presence of a fluoride ion source, such as, e.g., tetrabutylammonium fluoride.

The introduction of the azido group is accomplished after diazotization by reaction with sodium azide at room temperature.

Ether cleavages are performed according to processes that are common in literature. In this case, a selective cleavage can also be achieved in several groups that are present in the molecule. In this case, the ether is treated, for example, with boron tribromide in solvents such as dichloromethane at temperatures of between −100° C. up to the boiling point of the solvent, preferably at −78° C. It is also possible, however, to cleave the ether by sodium thiomethylate in solvents such as dimethylformamide. The temperature can be between room temperature and the boiling point of the solvent, preferably at 150° C.

The introduction of the alkenyl group is carried out with the corresponding vinyl compounds under the conditions of the Heck reaction. For the introduction of the ethinyl groups, the Songashira reaction is used.

As a leaving group R^{2′}, halogens such as fluorine, chlorine, bromine, iodine or O-mesylate, O-tosylate, O-triflate or O-nonaflate are suitable. The nucleophilic substitution for introducing ethinyl or ethenyl radicals is performed under the catalysis of transition metal complexes such as Pd(O), e.g., palladium tetrakis triphenylphosphine or Pd(2+), such as palladium-bis-tri-o-tolylphosphine-dichloride, nickel (II) or nickel (O) according to methods that are known in the literature, optionally in the presence of a base and optionally under co-catalysis of a salt, such as, for example, copper(I) iodide or lithium chloride.

As nucleophiles, for example, vinyl or ethinyl compounds, tin-organic compounds or zinc-organic compounds are suitable. The reaction can be performed in polar solvents, such as dimethylformamide, dimethylacetamide, N-methylpyrrolidone, acetonitrile, in hydrocarbons such as toluene or in ethers such as tetrahydrofuran, dimethoxyethane or diethyl ether. As bases, inorganic bases such as alkali or alkaline-earth hydroxides or bicarbonates, carbonates, phosphates or organic bases such as cyclic, alicyclic and aromatic amines, such as pyridine, triethylamine, DBU and Hünig base, are suitable, whereby in many cases, bases such as diethylamine or piperidine can also simultaneously be solvents. The application of pressure can be beneficial to the reaction.

If a trimethylsilylethinyl group is introduced, the trimethylsilyl group can by reaction with fluorides, such as, for example, potassium fluoride or tetrabutylammonium fluoride in solvents such as tetrahydrofuran, methylene chloride, or acetonitrile at temperatures of 0° C. up to the boiling point of the solvent.

An alkenyl group can also be introduced, however, by olefination reactions, such as, e.g., the Peterson olefination, the Wittig reaction or the Wittig-Homer reaction. To this end, the aldehyde is reacted with the anion that was already produced, e.g., a correspondingly substituted phosphonium salt or phosphonic acid ester in solvents such as toluene, tetrahydrofuran, diethyl ether or dimethoxyethane. As bases, e.g., alkali hydrides, alkali amides, alkali alcoholates, such as, for example, potassium tert-butylate, alkali and alkaline-earth carbonates or hydroxides optionally are suitable in the presence of phase-transfer catalysts, such as, e.g., crown ethers or else organic bases such as triethylamine diisopropylethylainine or diazabicycloundecane, optionally in the presence of salts such as lithium bromide.

The isomer mixtures can be separated into enantiomers or E/Z isomers according to commonly used methods, such as, for example, crystallization, chromatography or salt formation.

The production of the salts is carried out in the usual way by a solution of the compound of formula I being mixed with the equivalent amount or an excess of a base or acid, which optionally is in solution, and the precipitate being separated or the solution being worked up in the usual way.

If the production of the intermediate compounds is not described, the latter are known or can be produced analogously to known compounds or processes that are described here.

The intermediate compounds that are described are especially suitable for the production of benzoic acid amides according to the invention.

Especially suitable are those intermediate compounds of general formula II

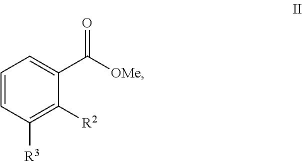

in which
R² and R³ mean hydrogen or the group XR¹¹,
X means C₁₋₆-alkyl, C₂₋₆-alkenyl or C₂₋₆-alkinyl,
R¹¹ means phenyl or pyridyl that is optionally substituted by C₁₋₆-alkoxy, whereby R² and R³ stand for hydrogen, although not simultaneously, as well as isomers and salts thereof.

These intermediate compounds are also subjects of this invention.

The intermediate products are themselves partially active and thus can also be used for the production of a pharmaceutical agent for treating tumors, psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, and injuries to nerve tissue.

The following examples explain the production of the compounds according to the invention without the scope of the claimed compounds being limited to these examples.

### EXAMPLE 1.0

### Production of (N-4-chlorophenyl)-2-(4-pyridylethyl)Benzoic Acid Amide

105 mg of 2-(4-pyridylethyl)benzoic acid methyl ester is mixed in 7.5 ml of toluene with 56 mg of 4-chloroaniline, cooled to 4° C. and mixed under argon and in a moisture-free environment with 0.22 ml of trimethylaluminum (2 m solution in hexane). Then, the mixture was heated for 2 hours to a bath temperature of 120° C. After cooling, it is mixed with 30 ml of a dilute sodium bicarbonate solution and extracted twice with 25 ml each of ethyl acetate. The organic phase is washed with water, dried, filtered and concentrated by evaporation. The residue is chromatographed on silica gel with ethyl acetate:cyclohexane=1:1 as an eluant. 133 mg (89% of theory) of (N-4-chlorophenyl)-2-(4-pyridylethyl)-benzoic acid amide is obtained as an oil.

Produced in a way similar to Example 1.0 are:

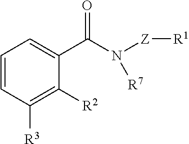

| | | | | | Melting Point |
|---|---|---|---|---|---|
| Example | —Z—R¹ | R⁷ | R² | R³ | ° C. |
| 1.1 | 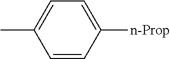 | H | 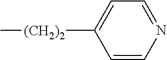 | H | |
| 1.2 | 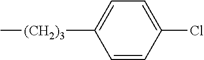 | H | 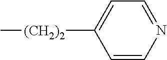 | H | |
| 1.3 | 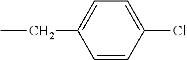 | H | 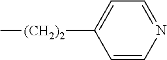 | H | 98-99 |
| 1.4 | 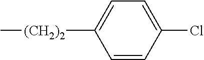 | H | 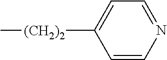 | H | Oil |
| 1.5 | 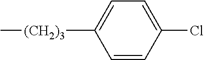 | H | 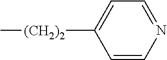 | H | Oil |
| 1.6 | 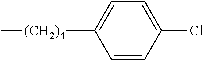 | H | 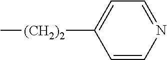 | H | Oil |
| 1.7 | 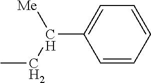 | H | 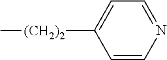 | H | |
| 1.8 | 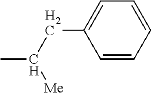 | H | 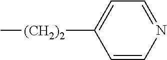 | H | Oil |
| 1.9 | 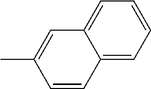 | H | 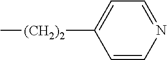 | H | |
| 1.10 | 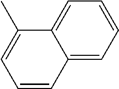 | H | 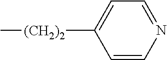 | H | |
| 1.11 | 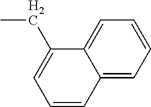 | H | 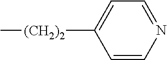 | H | Oil |
| 1.12 | 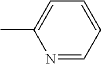 | H | 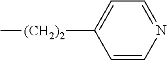 | H | |
| 1.13 | 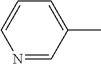 | H | 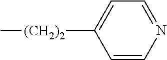 | H | |
| 1.14 | 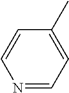 | H | 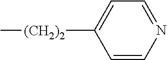 | H | |
| 1.15 | 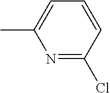 | H | 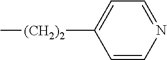 | H | |
| 1.16 | 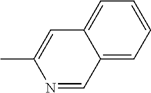 | H | 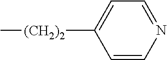 | H | |
| 1.17 | 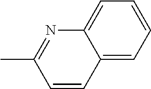 | H | 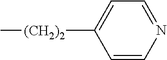 | H | Oil |
| 1.18 | 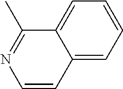 | H | 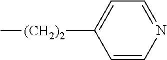 | H | |
| 1.19 | 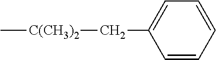 | H | 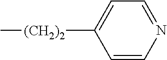 | H | |
| 1.20 | 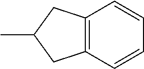 | H | 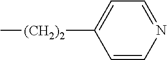 | H | 121-122 |
| 1.21 | 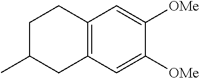 | H | 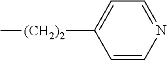 | H | |
| 1.22 | 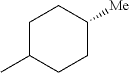 | H | 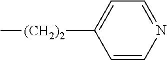 | H | |
| 1.23 | 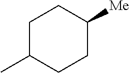 | H | 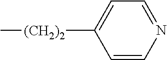 | H | |
| 1.24 | 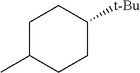 | H | 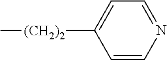 | H | 130-131 |
| 1.25 | 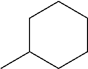 | H | 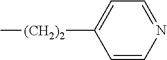 | H | |
| 1.26 | 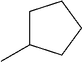 | H | 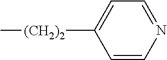 | H | |
| 1.27 | 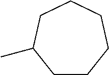 | H | 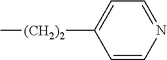 | H | |
| 1.28 | 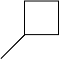 | H | 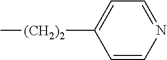 | H | |
| 1.29 | 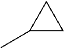 | H | 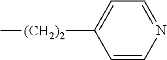 | H | |
| 1.30 | 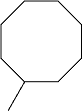 | H | 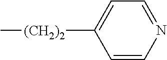 | H | |
| 1.31 | 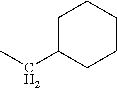 | H | 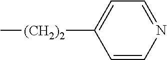 | H | 105-107 |
| 1.32 | —(CH₂)₁₁—Me | H | 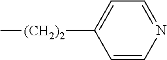 | H | |
| 1.33 | —(CH₂)₆—Me | H | 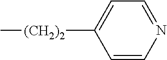 | H | |
| 1.34 | 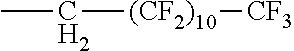 | H | 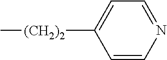 | H | |
| 1.35 | —(CH₂)₃—CF₃ | H | 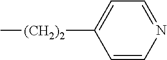 | H | |
| 1.36 | —(CH₂)₂-t-Bu | H | 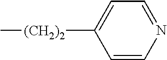 | H | |
| 1.37 | —(CH₂)₂-i-Prop | H | 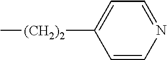 | H | |
| 1.38 | 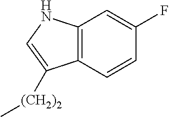 | H | 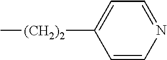 | H | |
| 1.39 | 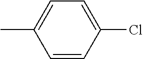 | H | 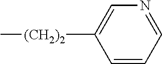 | H | 105.5 |
| 1.40 | 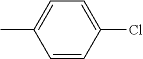 | H | 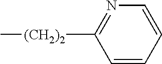 | H | 136.2 |
| 1.41 | 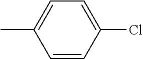 | H | 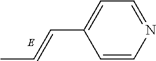 | H | Oil |
| 1.42 | 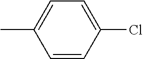 | H | 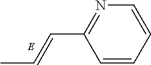 | H | 181.2 |
| 1.43 | 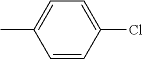 | H | 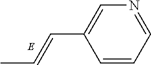 | H | Oil |
| 1.44 | 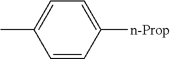 | H | 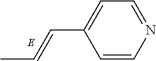 | H | |
| 1.45 | 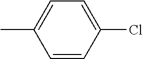 | H | 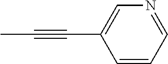 | H | 156.4 |
| 1.46 | 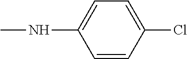 | H | 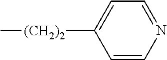 | H | |
| 1.47 | 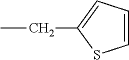 | H | 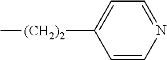 | H | |
| 1.48 | 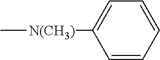 | H | 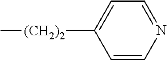 | H | |
| 1.49 | 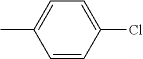 | H | H | 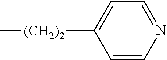 | 191.6 |
| 1.50 | 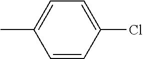 | H | H | 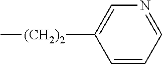 | 106.5 |
| 1.51 | 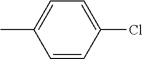 | H | H | 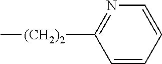 | 128.5 |
| 1.52 | 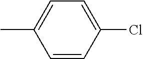 | H | H | 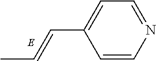 | 191.5 |
| 1.53 | 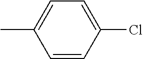 | H | H | 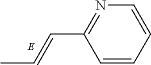 | 212.7 |
| 1.54 | 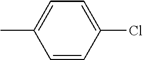 | H | H | 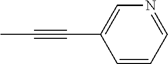 | 179 |
| 1.55 | 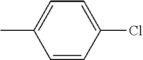 | H | H | 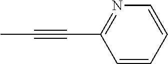 | >300 |
| 1.56 | 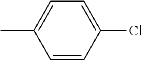 | H | H | 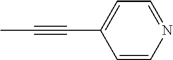 | 163.5 |
| 1.57 | 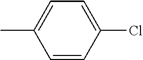 | H | 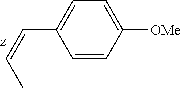 | H | 137.9 |
| 1.58 | 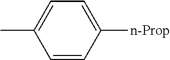 | H | 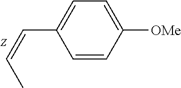 | H | 115-118 |
| 1.59 | 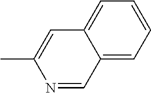 | H | 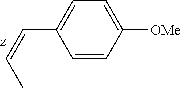 | H | 151-153 |
| 1.60 | 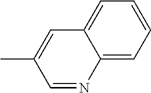 | H | 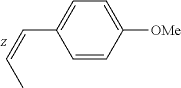 | H | |
| 1.61 | 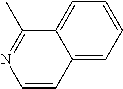 | H | 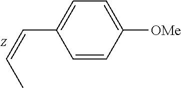 | H | |
| 1.62 | 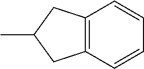 | H | 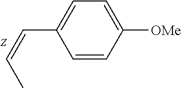 | H | |
| 1.63 | 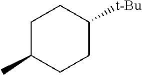 | H | 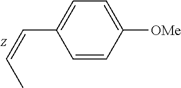 | H | |
| 1.64 | n-heptyl | H | 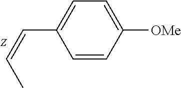 | H | Oil |
| 1.65 | 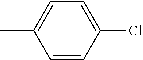 | H | 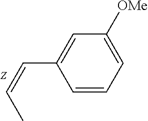 | H | 120.2 |
| 1.66 | 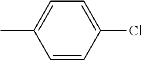 | H | 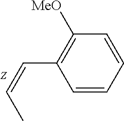 | H | 108.6 |
| 1.67 | 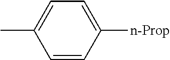 | H | 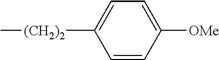 | H | 113.7 |
| 1.68 | 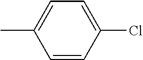 | H | 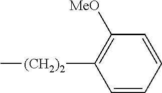 | H | |
| 1.69 | 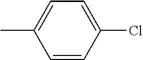 | H | 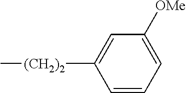 | H | |
| 1.70 | 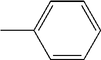 | H | 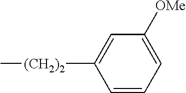 | H | |
| 1.71 | 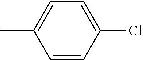 | H | 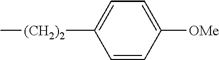 | H | |
| 1.72 | 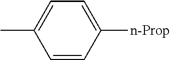 | H | H | 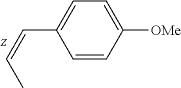 | |
| 1.73 | 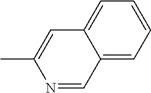 | H | H | 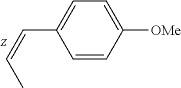 | |
| 1.74 | 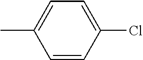 | H | H | 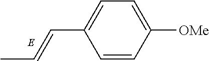 | 204.7 |
| 1.75 | 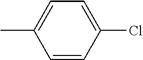 | H | H | 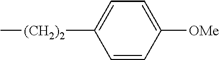 | >300 |
| 1.76 | 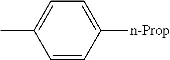 | H | H | 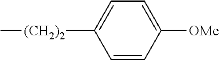 | |
| 1.77 | 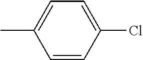 | H | H | 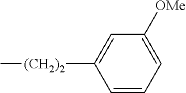 | 126.6 |
| 1.78 | 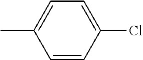 | H | H | 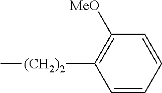 | 133.2 |
| 1.79 | 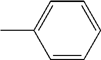 | H | H | 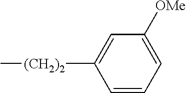 | 139.5 |
| 1.80 | 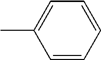 | H | H | 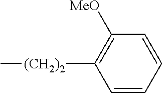 | 126.3 |
| 1.81 | 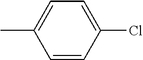 | H | 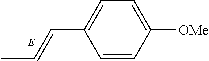 | H | |
| 1.82 | 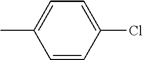 | H | 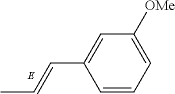 | H | 163.4 |
| 1.83 | 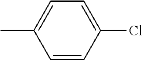 | H | 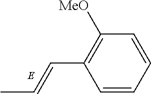 | H | 185.4 |
| 1.84 | 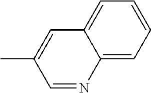 | H | 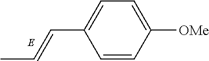 | H | Oil |
| 1.85 | 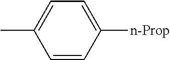 | H | 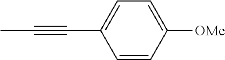 | H | |
| 1.86 | 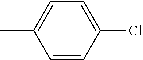 | H | 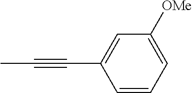 | H | 136.8 |
| 1.87 | 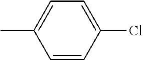 | H | 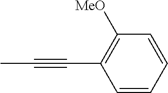 | H | 131.1 |
| 1.88 | 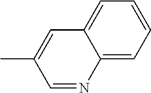 | H | 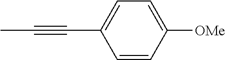 | H | 140.6 |
| 1.89 | 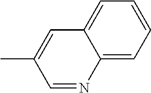 | H | 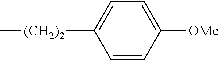 | H | Oil |
| 1.90 | 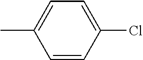 | H | H | 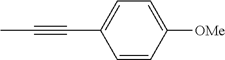 | 194.6 |
| 1.91 | 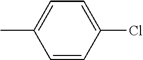 | H | H | 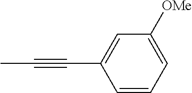 | 188.9 |
| 1.92 | 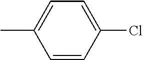 | H | H | 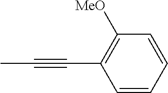 | |
| 1.93 | 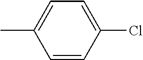 | H | 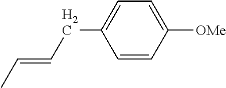 | H | 146.4 |
| 1.94 | 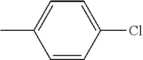 | H | 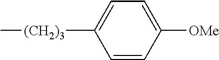 | H | |

### EXAMPLE 2.0

### Production of E-N-4-chlorophenyl-3-(2-pyridylethenyl)Benzoic Acid Amide

179 mg of N-4-chlorophenyl-3-(2-pyridylethinyl)benzoic acid amide is mixed in 7 ml of pyridine with 20 mg of palladium on calcium carbonate (5%), and it is hydrogenated for 1.5 hours under normal hydrogen pressure at room temperature. After the catalyst is suctioned off on diatomaceous earth, the filtrate is concentrated by evaporation. The residue is chromatographed on silica gel with ethyl acetate:hexane=1:1 as an eluant. 123 mg (68% of theory) of (Z)—N-4-chlorophenyl)-3-(2-pyridylethenyl)-benzoic acid amide is obtained as an oil.

Produced in a way similar to Example 2.0 are also the following compounds:

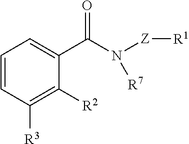

| | | | | | Melting Point |
|---|---|---|---|---|---|
| Example | —Z—R¹ | R⁷ | R² | R³ | ° C. |
| 2.1 | 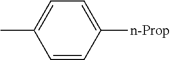 | H | H | 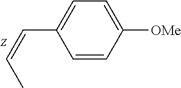 | |
| 2.2 | 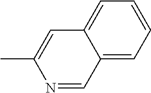 | H | H | 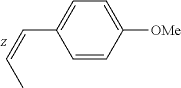 | |
| 2.3 | 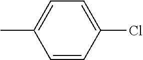 | H | H | 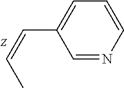 | 120.1 |
| 2.4 | 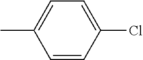 | H | H | 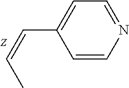 | 157.9 |
| 2.5 | 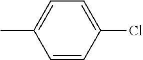 | H | H | 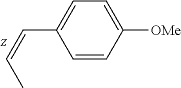 | 95.2 |
| 2.6 | 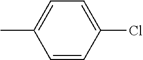 | H | H | 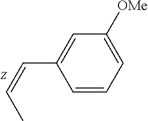 | 116.2 |
| 2.7 | 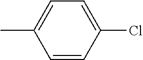 | H | H | 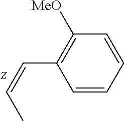 | 123 |

### EXAMPLE 3.0

### Production of (E)-N-4-chlorophenyl-3-(2-pyridylethenyl)Benzoic Acid Amide

120 mg of (Z)—N-4-chlorophenyl-3-(2-pyridylethenyl)benzoic acid amide is mixed in toluene with iodine and refluxed for 7 hours. After concentration by evaporation, the residue is chromatographed on silica gel with ethyl acetate:hexane=1:1 as an eluant. 60 mg (50% of theory) of (E)-N-4-chlorophenyl-3-(2-pyridylethenyl)benzoic acid amide with a melting point of 212.7° C. is obtained.

Similarly produced are:

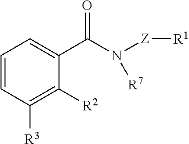

| | | | | | Melting Point |
|---|---|---|---|---|---|
| Example | —Z—R¹ | R⁷ | R² | R³ | ° C. |
| 3.1 | 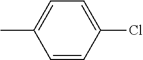 | H | H | 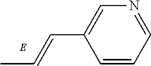 | 173.2 |
| 3.2 | 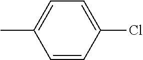 | H | H | 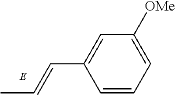 | 146.9 |
| 3.3 | 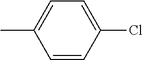 | H | H | 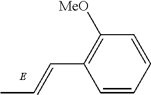 | 178 |

### EXAMPLE 4.0

### Production of N-(4-chlorophenyl)-2-(3-[4-hydroxyphenyl)propyl)]Benzoic Acid Amide

90 mg of N-(4-chlorophenyl)-2-(3-[4-methoxyphenyl)propyl)]benzoic acid amide is mixed in 8 ml of methylene chloride at −78° C. drop by drop with 1.2 ml of boron tribromide, and after the addition is completed, it is stirred overnight at room temperature. Then, it is mixed with water, the methylene chloride is distilled off in a vacuum, and the water is shaken out with ethyl acetate. The ethyl acetate phase is concentrated by evaporation, and the residue is chromatographed on silica gel with hexane:ethyl acetate=8:2 as an eluant. 24 mg (28% of theory) of N-(4-chlorophenyl)-2-(3-[4-hydroxyphenyl)propyl)]-benzoic acid amide is obtained.

Similarly produced are:

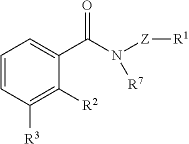

| | | | | | Melting Point |
|---|---|---|---|---|---|
| Example | —Z—R¹ | R⁷ | R² | R³ | ° C. |
| 4.1 | 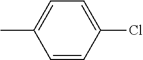 | H | 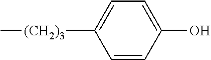 | H | |
| 4.2 | 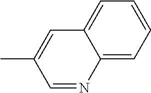 | H | 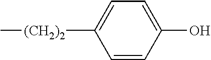 | H | |
| 4.3 | 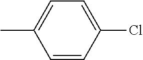 | H | H | 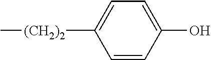 | |
| 4.4 | 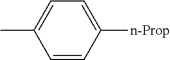 | H | 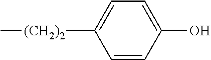 | H | |
| 4.5 | 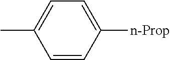 | H | H | 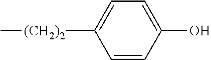 | |
| 4.6 | 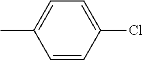 | H | 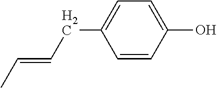 | H | |
| 4.7 | 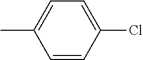 | H | OH | 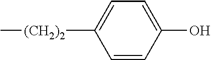 | 164.1 |
| 4.8 | 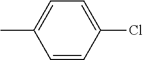 | H | OH | 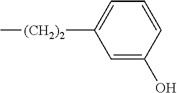 | 115.3 |
| 4.9 | 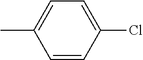 | H | OH | 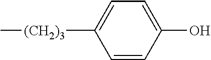 | 137.4 |
| 4.10 | 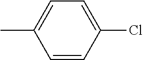 | H | OH | 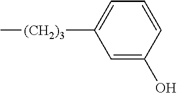 | Oil |
| 4.11 | 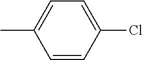 | H | OH | 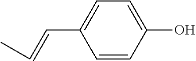 | 203.7 |
| 4.12 | 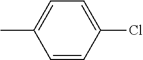 | H | OH | 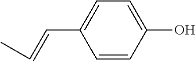 | |

### EXAMPLE 5.0

### Production of N-(4-chlorophenyl)-3-(4-methoxystyryl)Salicylic Acid Amide

904 mg of 4-methoxy-2′-hydroxystyrene is introduced into 40 ml of toluene and mixed at 4° C. with 4 ml of a solution of boron trichloride (1 mol in hexane). It is then stirred at room temperature for 1 hour, mixed with 614 mg of 4-chlorophenyl isocyanate and heated for 1.5 hours to 120° C. Then, it is mixed with 5 ml of methanol and concentrated by evaporation. The residue is chromatographed twice on silica gel, first with ethyl acetate:hexane=1:1, and a second time with toluene:ethyl acetate=100:3.5 as an eluant. 150 mg (10% of theory) of N-(4-chlorophenyl)-3-(4-methoxystyryl)salicylic acid amide is obtained as an oil.

Similarly produced from the corresponding starting materials are:

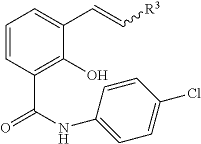

| | | Melting Point |
|---|---|---|
| Example | R³ | ° C. |
| 5.1 | 3-MeO—Ph | 116.3 |
| 5.2 | 3-MeO—Ph—CH₂ | |
| 5.3 | 4-MeO—Ph—CH₂ | 163.6 |

### Production of the Intermediate Compounds

### EXAMPLE Z-5.0

### Production of 4-methoxy-2′-hydroxystyrene

2.44 g of salicyl aldehyde is mixed in 200 ml of toluene first with 12.5 g of 4-methoxy-benzyltriphenylphosphonium chloride. 2.24 g of potassium-tert-butylate is then added while being cooled with ice. It is then stirred first for 1 hour at this temperature and then for 3.5 hours at room temperature. After mixing with 100 ml of water and acidification with 1N hydrochloric acid, it is extracted three times with 50 ml of ethyl acetate. The collected organic phase is washed with saturated sodium chloride solution, dried, filtered and concentrated by evaporation. The residue is chromatographed on silica gel with ethyl acetate:hexane=2:8 as an eluant. 3.1 g (68% of theory) of 4-methoxy-2′-hydroxystyrene is obtained.

Similarly produced are also the following compounds:

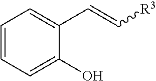

| | | Melting Point |
|---|---|---|
| Example | R³ | ° C. |
| Z-5.1 | 3-MeO—Ph | Oil |
| Z-5.2 | 3-MeO—Ph—CH₂ | Oil |
| Z-5.3 | 4-MeO—Ph—CH₂ | Oil |

### EXAMPLE 6.0

### Production of N-(4-chlorophenyl)-3-(4-methoxyphenethyl)Salicylic Acid Amide

813 mg of 4-methoxy-2′-hydroxy 1,2-diphenylethane is reacted analogously to Example Z-5.0. The residue that is obtained after the working-up that is described there is chromatographed on silica gel with ethyl acetate:hexane=1:1 as an eluant, and the corresponding fractions are concentrated by evaporation and stirred with ethyl acetate/hexane in crystalline form. 375 mg (27.6% of theory) of N-(4-chlorophenyl)-3-(4-methoxyphenylethyl)salicylic acid amide with a melting point of 141° C. is obtained.

Similarly produced are also the following compounds:

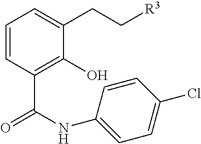

| | | Melting Point |
|---|---|---|
| Example | R³ | ° C. |
| 6.1 | 3-MeO—Ph | 130.2 |
| 6.2 | 3-MeO—Ph—CH₂ | Oil |
| 6.3 | 4-MeO—Ph—CH₂ | 158 |

### Production of the Intermediate Compounds

### EXAMPLE Z-6.0

### Production of 4-methoxy-2′-hydroxy-1,2-diphenylethane

905 mg of 4-methoxy-2′-hydroxystyrene is mixed in 50 ml of ethanol with 1.3 g of palladium on carbon (10) and hydrogenated for 70 minutes at room temperature under normal hydrogen pressure. After the catalyst is suctioned off and after concentration by evaporation, 830 mg of 4-methoxy-2′-hydroxy 1,2-diphenylethane is obtained.

Similarly produced are also the following compounds:

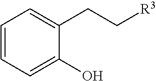

| | | Melting Point |
|---|---|---|
| Example | R³ | ° C. |
| Z-6.1 | 3-MeO—Ph | Oil |
| Z-6.2 | 3-MeO—Ph—CH₂ | Oil |
| Z-6.3 | 4-MeO—Ph—CH₂ | |

### EXAMPLE 7.0

### Production of Intermediate Products

The examples below explain the production of the intermediate products according to the invention that are especially suitable for the production of the compounds of general formula I according to the invention, without the invention being limited to these examples.

### Method A

Production of 2-(4-pyridiylethenyl)Benzoic Acid Methyl Ester

A mixture of 2.10 g of 2-iodobenzoic acid methyl ester and 0.97 g of 4-vinylpyridine in 24 ml of dimethylformamide is mixed with 1.04 g of triethylamine and 40 mg of palladium(II) acetate as well as 24 mg of tri-o-tolylphosphine under argon, and it is heated for 5 hours in a glass pressure vessel to 100° C. After concentration by evaporation in a vacuum, the residue is chromatographed on silica gel with hexane:ethyl acetate=1:1 as an eluant.

1.8 g (94% of theory) of 2-(4-pyridiylethenyl)-benzoic acid methyl ester is obtained.

### Method B

Production of 2-(4-pyridylethinyl)Benzoic Acid Methyl Ester

2.10 g of 2-iodobenzoic acid methyl ester is mixed in 25 ml of dimethylformamide under argon with 2.94 g of triethylamine, 179 mg of bis(triphenylphosphine)palladium(II) chloride, 111 mg of copper(I) iodide and 900 mg of 4-ethinylpyridine, and it is heated in a glass pressure vessel for 3.5 hours to a bath temperature of 80° C. After concentration by evaporation in a vacuum, the residue is chromatographed on silica gel with hexane:acetone=1:1 as an eluant.

1.08 g (45% of theory) of 2-(4-pyridiylethinyl)-benzoic acid methyl ester is obtained.

### Method C

Production of 2-(4-pyridylethyl)Benzoic Acid Methyl Ester

237 mg of 2-(4-pyridylethinyl)benzoic acid methyl ester, is mixed in 30 ml of ethanol with 200 mg of palladium on carbon (10%) and hydrogenated at normal pressure and at room temperature for 20 minutes. Then, catalyst is suctioned off on diatomaceous earth, and the filtrate is concentrated by evaporation. 220 mg of 2-(4-pyridylethyl)benzoic acid methyl ester is obtained.

Instead of the ethinyl compound, the corresponding ethenyl compound can also be used.

### Method D

According to the method that is described in Example 2.0, the corresponding esters can also be converted into the Z compounds.

### Method E

According to the method that is described in Example 3.0, in the case of the esters, the E compounds can also be produced from the corresponding Z compound.

### Method F

According to the method that is described in Method B, 2-trimethylsilylethinylbenzoic acid methyl ester can also be produced from 2-iodobenzoic acid ethyl ester with ethinyltrimethylsilane in 83% yield.

### Method G

464 mg of 2-trimethylsilylethinylbenzoic acid methyl ester is mixed in 15 ml of absolute methylene chloride with 2.75 ml of tetrabutylammonium fluoride (1 M in tetrahydrofuran) and stirred for 2.5 hours at room temperature. After washing with dilute ammonia, the organic phase is dried, filtered and concentrated by evaporation and used without further purification in the next stage.

### Method H

440 mg of 2-ethinylbenzoic acid methyl ester is reacted with 1.94 g of 3-iodoanisole according to Method B, and 680 mg (55.3% of theory) of 2-carbethoxymethyl-3′-methoxydiphenylacetylene is produced after column chromatography on silica gel with ethyl acetate:hexane=2.8 as an eluant.

Similarly produced are also the following compounds:

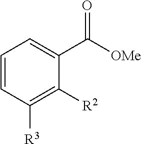

| Example | R² | R³ | Method |
|---|---|---|---|
| 7.0 | 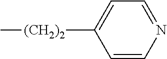 | H | C |
| 7.1 | 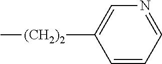 | H | C |
| 7.2 | 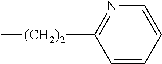 | H | C |
| 7.3 | 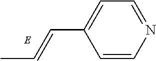 | H | A |
| 7.4 | 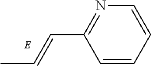 | H | A |
| 7.5 | 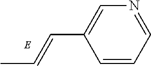 | H | E |
| 7.6 | 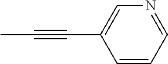 | H | B |
| 7.7 | 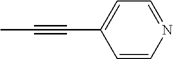 | H | B |
| 7.8 | 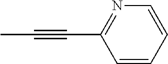 | H | B |
| 7.9 | H | 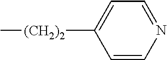 | C |
| 7.10 | H | 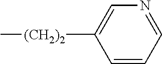 | C |
| 7.11 | H | 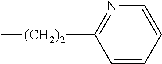 | C |
| 7.12 | H | 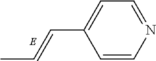 | A |
| 7.13 | H | 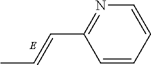 | A |
| 7.14 | H | 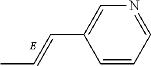 | E |
| 7.15 | H | 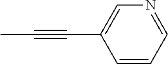 | B |
| 7.16 | H | 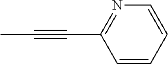 | B |
| 7.17 | H | 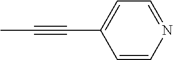 | B |
| 7.18 | H | 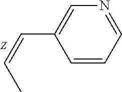 | D |
| 7.19 | 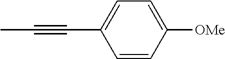 | H | B |
| 7.20 | 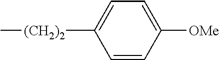 | H | C |
| 7.21 | 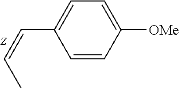 | H | D |
| 7.22 | 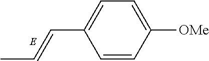 | H | A |
| 7.23 | 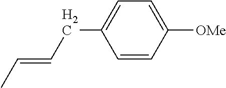 | H | A |
| 7.24 | 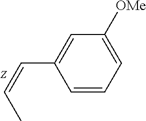 | H | D |
| 7.25 | 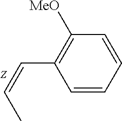 | H | D |
| 7.26 | 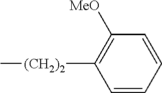 | H | C |
| 7.27 | 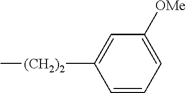 | H | C |
| 7.28 | 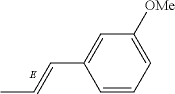 | H | E |
| 7.29 | 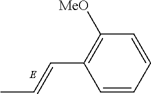 | H | A |
| 7.30 | 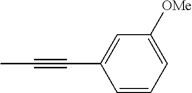 | H | F-H |
| 7.31 | 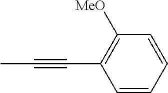 | H | B |
| 7.32 | 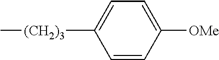 | H | C |
| 7.33 | H | 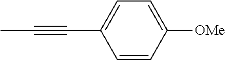 | B |
| 7.34 | H | 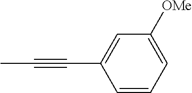 | F-H |
| 7.35 | H | 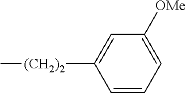 | C |
| 7.36 | H | 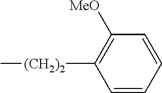 | C |
| 7.37 | H | 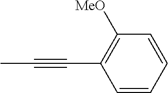 | F-H |
| 7.38 | H | 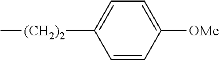 | C |

The sample applications below explain the biological action and the use of the compounds according to the invention without the latter being limited to the examples.

### Solutions Required for the Tests

Stock solutions
Stock solution A: 3 mmol of ATP in water, pH 7.0 (−70° C.)
Stock solution B: g-33P-ATP 1 mCi/100 μl
Stock solution C: poly-(Glu4Tyr) 10 mg/ml in water
   Solution for dilutions
Substrate solvent: 10 mmol of DTT, 10 mmol of manganese chloride, 100 mmol of magnesium chloride
Enzyme solution: 120 mmol of tris/HCl, pH 7.5, 10 μM of sodium vanadium oxide

### Sample Application 1

### Inhibition of the KDR- and FLT-1 Kinase Activity in the Presence of the Compounds According to the Invention

In a microtiter plate (without protein binding) that tapers to a point, 10 μl of substrate mix (10 μl of volume of ATP stock solution A+25 μCi of g-33P-ATP (about 2.5 μl of stock solution B)+30 μl of poly-(Glu4Tyr) stock solution C+1.21 ml of substrate solvent), 10 μl of inhibitor solution (substances corresponding to the dilutions, 3% DMSO in substrate solvent as a control) and 10 μl of enzyme solution (11.25 μg of enzyme stock solution (KDR or FLT-1 kinase) are added at 4° C. in 1.25 ml of enzyme solution (dilute). It is thoroughly mixed and incubated for 10 minutes at room temperature. Then, 10 μl of stop solution (250 mmol of EDTA, pH 7.0) is added, mixed, and 10 μl of the solution is transferred to a P 81 phosphocellulose filter. Then, it is washed several times in 0.1 M phosphoric acid. The filter paper is dried, coated with Meltilex and measured in a microbeta counter.

The IC50 values are determined from the inhibitor concentration, which is necessary to inhibit the phosphate incorporation to 50% of the uninhibited incorporation after removal of the blank reading (EDTA-stopped reaction).

The results of the kinase inhibition IC50 in μM are presented in the table below:

| Example No. | VEGFR I (FLT) | VEGFR II (KDR) |
|---|---|---|
| 1.60 | 2 | 0.5 |
| 1.31 | 0.2 | 0.4 |
| 1.89 | 2 | 0.3 |
| 1.54 | 0.05 | 0.5 |
| 1.57 | 0.2 | 0.2 |
| 1.64 | 0.2 | 0.3 |
| 1.67 | KH | 5 |
| 1.1 | 0.2 | 0.2 |

KH = No inhibition

1. Compounds of general formula I 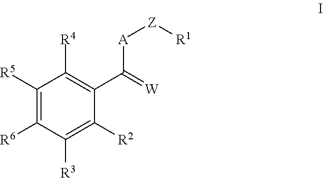
in which
A stands for the group ═NR⁷,
W stands for oxygen, sulfur, two hydrogen atoms or the group ═NR⁸,
Z stands for a bond, the group ═NR¹⁰ or ═N—, for branched or unbranched C₁₋₁₂-alkyl or for the group 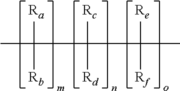
m, n and o stand for 0-3,
Rₐ, R_{b}, R_{c}, R_{d}, Rₑ, R_{f}, independently of one another, stand for hydrogen, fluorine, C₁₋₄-alkyl or the group ═N¹⁰, and/or Rₐ and/or R_{b} can form a bond with R_{c} and/or R_{d} or R_{c} can form a bond with Rₑ and/or R_{f}, or up to two of radicals Rₐ-R_{f} can close a bridge with up to 3 C atoms each to form R¹ or to form R⁷,
R¹ stands for branched or unbranched C₁₋₆-alkyl, C₂₋₁₂-alkenyl or C₃₋₁₂-alkinyl that is optionally substituted in one or more places with halogen or C₁₋₆-alkyl; or for C₃₋₁₀-cycloalkyl or C₃₋₁₀-cycloalkenyl that is optionally substituted in one or more places with halogen or C₁₋₆-alkyl; or for aryl or hetaryl that is unsubstituted or that is optionally substituted in one or more places with halogen, C₁₋₆-alkyl, C₁₋₆-alkoxy, or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkinyl,
R¹¹ means monocyclic aryl, bicyclic aryl or heteroaryl that is unsubstituted or that is optionally substituted in one or more places with halogen, C₁₋₆-alkyl, C₁₋₆-alkoxy, or hydroxy,
R⁴, R⁵ and R⁶ stand for hydrogen, halogen, or C₁₋₆-alkoxy, C₁₋₆-alkyl or C₁₋₆-carboxyalkyl that is unsubstituted or that is optionally substituted in one or more places with halogen,
or R⁴ and R⁵ together form the group 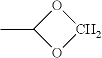
R⁷ stands for hydrogen or C₁₋₆-alkyl or forms a bridge with up to 3 ring members with Rₐ-R_{f} from Z or to form R¹,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, and R¹ must not be thiazole, as well as isomers and salts thereof.

2. Compounds of general formula I, according to claim 1, in which
A stands for the group ═NR⁷,
W stands for oxygen, sulfur or two hydrogen atoms,
Z stands for a bond, the group ═NR¹⁰ or for branched or unbranched C₁₋₁₂-alkyl,
R¹ stands for branched or unbranched C₁₋₆-allyl that is optionally substituted in one or more places with halogen or C₁₋₆-alkyl; or for C₃₋₁₀-cycloalkyl that is optionally substituted in one or more places with halogen or C₁₋₆-alkyl; or for phenyl, pyridyl, naphthyl, quinolyl, isoquinolyl, indanyl, tetralinyl, indolyl, thienyl, indazolyl or benzothiazolyl that is unsubstituted or that is optionally substituted in one or more places with halogen, C₁₋₆-alkyl, C₁₋₆-alkoxy or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkinyl,
R¹¹ means phenyl, pyrimidinyl or pyridyl that is unsubstituted or that is optionally substituted in one or more places with halogen, C₁₋₆-alkoxy or hydroxy,
R⁴, R⁵, R⁶ and R⁷ stand for hydrogen,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, as well as isomers and salts thereof.

3. Compounds of general formula I, according to claims 1 and 2, in which
A stands for the group ═NR⁷,
W stands for oxygen, or for one or two hydrogen atoms,
Z stands for a bond, the group ═NR¹⁰ or for branched or unbranched C₁₋₁₂-alkyl,
R¹ stands for branched or unbranched C₁₋₆-alkyl; or for C₃₋₁₀-cycloalkyl that is optionally substituted in one or more places with halogen or C₁₋₆-alkyl; or for phenyl, pyridyl, naphthyl, quinolyl, indenyl, tetralinyl, indolyl, thienyl, indazolyl, or benzothiazolyl that is unsubstituted or that is optionally substituted in one or more places with halogen, C₁₋₆-alkyl, C₁₋₆-alkoxy or C₁₋₆-alkyl or C₁₋₆-alkoxy that is substituted in one or more places with halogen,
R² and R³ stand for hydrogen, an OH group or the group XR¹¹,
X stands for C₂₋₆-alkyl, C₂₋₆-alkenyl or C₂₋₆-alkenyl,
R¹¹ stands for phenyl, pyrimidinyl or pyridyl that is unsubstituted or that is optionally substituted in one or more places with halogen, C₁₋₆-alkoxy or hydroxy,
R⁴, R⁵, R⁶ and R⁷ stand for hydrogen,
R⁸ and R¹⁰ stand for hydrogen or C₁₋₆-alkyl, whereby R² and R³ stand for hydrogen, although not simultaneously, and if R² stands for an OH group, R³ does not stand for hydrogen, and if R³ stands for an OH group, R² does not stand for hydrogen, as well as isomers and salts thereof.

4. Use of the compounds of general formula I, according to claims 1 to 3, for the production of a pharmaceutical agent for treating tumors, psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, and injuries to nerve tissue.

5. Pharmaceutical agents that contain at least one compound according to claims 1 to 3.

6. Pharmaceutical agents according to claim 5, for treating tumors, psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, and injuries to nerve tissue.

7. Compounds, according to claims 1 to 3, with suitable formulations and vehicles.

8. Use of the compounds of formula I according to claims 1 to 3 as inhibitors of the tyrosine kinase KDR and FLT.

9. Use of the compounds of general formula I, according to claims 1 to 3, in the form of a pharmaceutical preparation for enteral, parenteral and oral administration.

10. Intermediate compounds of general formula II 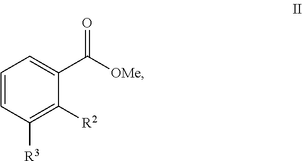
in which
R² and R³ mean hydrogen or the group X¹¹,
X means C₁₋₆-alkyl, C₂₋₆-alkenyl or C₂₋₆-alkinyl,
R¹¹ means phenyl or pyridyl that is optionally substituted by C₁₋₆-alkoxy, whereby R² and R³ stand for hydrogen, although not simultaneously, as well as isomers and salts thereof, as intermediate products for the production of the compounds of general formula I.

11. Compounds of general formula II, according to claim 10, for the production of a pharmaceutical agent for treating tumors, psoriasis; arthritis, such as rheumatoid arthritis, hemangioma, angiofibroma; eye diseases, such as diabetic retinopathy, neovascular glaucoma; renal diseases, such as glomerulonephritis, diabetic nephropathy, malignant nephrosclerosis, thrombic microangiopathic syndrome, transplant rejections and glomerulopathy; fibrotic diseases, such as cirrhosis of the liver, mesangial cell proliferative diseases, arteriosclerosis, and injuries to nerve tissue.
